(19) United States
(12) Patent Application Publication
(10) Pub. No.: US 2012/0263660 A1
(43) Pub. Date: Oct. 18, 2012

(54) Hair Loss Treatment

(75) Inventors: Randice Lisa Altschul, Cliffside Park, NJ (US); Neil David Theise, New York, NY (US); Myron Rapkin, Indianapolis, IN (US); Rebecca O'Brien, Shell Knob, MO (US)

(73) Assignee: POP TEST CORTISOL LLC, Cliffside Park, NJ (US)

(21) Appl. No.: 13/447,345

(22) Filed: Apr. 16, 2012

Related U.S. Application Data
(60) Provisional application No. 61/517,343, filed on Apr. 18, 2011.

(51) Int. Cl. A61K 31/58; A61Q 1/12; A61Q 19/04; A61Q 3/02; A61Q 9/02; A61Q 5/06; A61Q 5/02; A61Q 5/12; A61Q 19/10; A61Q 17/04; A61Q 5/04; A61Q 15/00; A61Q 19/00; A61K 8/67; A61K 8/66; A61K 8/92; A61P 31/00; A61P 17/10; A61Q 19/02; A61P 17/14; A61P 17/00; A61Q 5/10; A61Q 9/04; A61K 38/43; A61K 36/886; A61K 8/97; A61K 31/704; B65D 69/00; A61K 8/63

(52) U.S. Cl. 424/59; 514/172; 424/69; 424/61; 424/65; 424/62; 514/174; 8/405; 424/94.1; 424/63; 424/744; 514/33; 206/570

(57) ABSTRACT

This invention relates to the use of cortisol blockers (glucocorticoid receptor [GR] antagonists) for the prevention or treatment of stress induced hair loss.

### CROSS-REFERENCE TO RELATED APPLICATIONS

This application claims benefit under 35 U.S.C. §119(e) to U.S. Provisional Patent Application 61/517,343 filed Apr. 18, 2011, the disclosure of which is incorporated herein in its entirety.

### BACKGROUND OF THE INVENTION

1. Field of Invention

This invention relates to the use of cortisol blockers (glucocorticoid receptor [GR] antagonists) for the prevention or treatment of stress induced hair loss.

2. Description of Related Art

An intense psychic shock can exert pronounced effects on the hair. Both graying and generalized loss of hair is observed. It is furthermore shown that chronic stress exerts an inhibitory effect on hair growth.

The present invention relates to the use of cortisol blockers (glucocorticoid receptor [GR] antagonists) for the prevention or treatment of stress-induced hair loss. Such stressors could be, but are not limited to, chemotherapy or traumatic stressful events.

In human hair follicles corticotropin-releasing factor (CRF), adrenocorticotropic hormone (ACTH) and cortisol are produced and can be measured. It is suggested that stress hormones can enter the follicles and have a direct and local effect on the hair.

Mice that over-express CRF have been characterized as a model of chronic stress. These animals show features of Cushing's syndrome. One of the phenotypic characteristics is a bilateral symmetric hair loss (alopecia) in adulthood. Adrenalectomy can reverse this alopecia which suggests a direct role of the stress hormone cortisol.

Typical methods of inducing hair growth focus on manipulating cell death to prevent premature baldness or graying of hair. Despite the vigorous research and development focusing on hair growth agents, the hair fostering action, namely hair loss prevention and hair growth effects and the like, of conventional hair growth agents has not always been adequate. A number of hair growth stimuli were developed from growth factors, hormones, plant extracts, and a combination thereof with limited success achieved so far.

Currently available treatments acknowledged by dermatologists include orally administered PROPECIA® and externally applied Rogaine® (Messenger A G, Rundegren J. Minoxidil: mechanisms of action on hair growth. Br J. Dermatol. 2004 February; 150(2): 186-94. Review). PROPECIA® is the first FDA-approved pill demonstrated to treat male pattern hair loss on the vertex (top of head) and anterior mid-scalp area (middle front of head) in men. However, it is not effective when used by females and may have a side effect of impairing male sexual function. ROGAINE® (also known as minoxidil) is used topically to stimulate hair growth on the bald spot of the back of the head in men. In women, ROGAINE® can increase hair growth in the forehead areas. Yet, an increase in the absorption of minoxidil from the scalp can occur in patients with inflamed or abnormal scalps, leading to side effects, including a fall in blood pressure, an increase in the heart rate, and weight gain (fluid retention). As a result, ROGAINE® is usually used with caution in those with high blood pressure. Also, the alcohol base in ROGAINE® can irritate the eyes.

There is a long-felt and still growing need for a method of prevention or treatment of stress-induced hair loss. The present invention satisfies that need.

All references cited herein are incorporated herein by reference in their entireties.

### BRIEF SUMMARY OF THE INVENTION

The invention provides a topical composition for treating or preventing stress induced hair loss in a patient comprising: a hair growth stimulating agent which is at least one GR antagonist in a therapeutically effective amount; a cosmetically or pharmaceutically acceptable excipient, carrier or vehicle, wherein the composition has the effect of treating or preventing stress induced hair loss in the patient.

The invention further provides a composition of the invention wherein the GR antagonist is selected from the group consisting of ORG 34517, 11-(substituted phenyl)-estra-4,9-diene derivatives, and 11-(substituted phenyl)-estra-4,9-diene derivatives of formula I

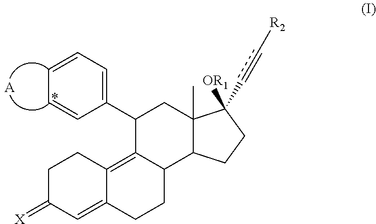

wherein A is a residue of a 5- or 6-membered ring containing 2 heteroatoms which are not connected to each other and independently selected from O and S, the ring being optionally substituted with one or more halogen atoms, or A is a residue of a 5- or 6-membered ring wherein no double C—C bonds are present, containing 1 heteroatom selected from O and S, which heteroatom is connected to the phenyl group at the position indicated with an asterisk, the ring being optionally substituted with one or more halogen atoms; R1 is H or 1-oxo(1-4C)alkyl; R2 is H, (1-8C)alkyl, halogen or CF3; X is selected from (H,OH), O, and NOH; and the interrupted line represents an optional bond, and combinations thereof.

The invention further provides a composition of the invention wherein the hair is selected from the group consisting of head hair, scalp hair, eyelashes, eyebrows, mustaches, beards, chest, legs, arms, and combinations thereof.

The invention further provides a composition of the invention in a form selected from the group consisting of application stick, mascara, eyebrow coloring, eyeliner, eye shadow or other eye lid cosmetics, facial or body powder, sun tanning lotions and creams and sprays, sunscreens and blocks, nail polish, mousse, sprays, styling gels, nail conditioner, whether in the form of creams, lotions, gels, ointments, emulsions, colloids, solutions, suspensions, compacts, solids, pencils, brushes, spray-on formulations, brush-on formulations and the like, bath and shower gels, shampoos, conditioners, cream rinses, hair dyes and coloring products, leave-on conditioners, lip balms, skin conditioners, hair sprays, soaps, body scrubs, exfoliants, astringents, depilatories and permanent or temporary waving solutions, antidandruff formulations, antisweat and antiperspirant compositions, shaving, preshaving and after shaving products, moisturizers, deodorants, cold creams, cleansers, skin gels, rinses, whether in solid, powder, liquid, cream, gel, ointment, lotion, emulsions, colloids, solutions, suspensions, or other form, gels, patches, creams, sprays, ointments, lotions, emulsions, colloids, solutions, suspensions, powders and the like, and combinations thereof.

The invention further provides a composition of the invention further comprising at least one other ingredient selected from the group consisting of solvents, thickeners or gelling agents, coloring materials which are soluble in the medium of the composition, fillers, pigments, antioxidants, preservatives, fragrances, electrolytes, neutralizing agents, film-forming polymers, UV blocking agents, cosmetic and pharmaceutical active principles other than GR antagonists, and combinations thereof.

The invention further provides a composition of the invention wherein said at least one additional ingredient is a cleaning agent, hair conditioning agent, skin conditioning agent, hair styling agent, antidandruff agent, hair growth promoter, perfume, sunscreen, sunblock, pigment, moisturizer, film former, hair color, make-up agent, detergent, pharmaceutical, thickening agent, emulsifier, humectant, emollient, antiseptic agent, deodorant active, dermatologically acceptable carrier, surfactant, abrasive, absorbent, fragrance, coloring/colorant, essential oil, skin sensate, astringent, anti-acne agent, anti-caking agent, antifoaming agent, antimicrobial, antioxidant, binder, biological additive, enzyme, enzyme inhibitor, enzyme activator, coenzyme, botanical extract, ceramide, addition peptide, buffering agent, bulking agent, chelating agent, cosmetic biocide, denaturant drug astringent, external analgesic, polymer, quat, substantivity increasing agent, opacifying agent, pH adjuster, propellant, reducing agent, sequestrant, skin bleaching agent, skin lightening agent, skin-conditioning agent, skin soothing agent, skin healing agent, aloe vera, pantothenic acid and derivative thereof, allantoin, bisabolol, dipotassium glycyrrhizinate, skin treating agent, thickener, or a vitamin or derivative thereof and mixtures or combinations thereof.

The invention further provides a composition of the invention further comprising a plurality of additional ingredients and wherein at least one of said additional ingredients is a dermatologically acceptable carrier.

The invention provides a delivery device for the targeted application to the hair of a mammal a therapeutically effective amount of the composition of the invention.

The invention further provides a delivery device of the invention wherein the device is for the application of the composition to eyelashes, eyebrows, and/or scalp.

The invention provides a method for treating or preventing stress induced hair loss wherein said method comprises administering, to a patient in need of such therapy, at least one GR antagonist in a therapeutically effective amount.

The invention further provides a method of the invention wherein the at least one GR antagonist is in a pharmaceutical preparation.

The invention further provides a method of the invention wherein the GR antagonist is selected from the group consisting of ORG 34517, 11-(substituted phenyl)-estra-4,9-diene derivatives, and 11-(substituted phenyl)-estra-4,9-diene derivatives of formula I

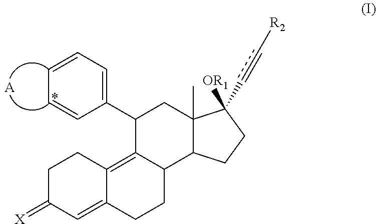

wherein A is a residue of a 5- or 6-membered ring containing 2 heteroatoms which are not connected to each other and independently selected from O and S, the ring being optionally substituted with one or more halogen atoms, or A is a residue of a 5- or 6-membered ring wherein no double C—C bonds are present, containing 1 heteroatom selected from O and S, which heteroatom is connected to the phenyl group at the position indicated with an asterisk, the ring being optionally substituted with one or more halogen atoms; R1 is H or 1-oxo(1-4C)alkyl; R2 is H, (1-8C)alkyl, halogen or CF3; X is selected from (H,OH), O, and NOH; and the interrupted line represents an optional bond.

The invention further provides a method of the invention wherein the hair is selected from the group consisting of head hair, scalp hair, eyelashes, eyebrows, mustaches, beards, chest, legs, arms, and combinations thereof.

The invention provides a method of treating or preventing hair loss secondary to stress in a patient in need thereof, comprising administering at least one GR antagonist in a therapeutically effective amount.

The invention further provides a method of the invention wherein the stress is selected from the group consisting of chemotherapy, traumatic events, and combinations thereof.

The invention further provides a method of the invention wherein the hair is selected from the group consisting of head hair, scalp hair, eyelashes, eyebrows, mustaches, beards, chest, legs, arms, and combinations thereof.

The invention provides a kit for treating or preventing stress induced hair loss comprising: (a) a pharmaceutical composition comprising at least one GR antagonist in a therapeutically effective amount; and (b) at least one blister package; a lidded blister; a blister card or packet; a clamshell; an intravenous (IV) package, IV packette or IV container; a tray or a shrink wrap comprising the pharmaceutical composition of (a) and instructions for use of the pharmaceutical composition for treating or preventing stress induced hair loss.

The invention provides a product of manufacture for treating or preventing stress induced hair loss comprising a blister package; a lidded blister; a blister card or packet; a clamshell; an intravenous (IV) package, IV packette or IV container; a tray or a shrink wrap comprising a pharmaceutical composition comprising at least one GR antagonist, and instructions for use of the pharmaceutical composition for treating or preventing stress induced hair loss.

The invention provides a topical composition for treating or preventing stress induced keratinous tissue damage in a patient comprising: a keratinous tissue growth stimulating agent which is at least one GR antagonist in a therapeutically effective amount; a cosmetically or pharmaceutically acceptable excipient, carrier or vehicle, wherein the composition has the effect of treating or preventing stress induced keratinous tissue damage in the patient.

The invention further provides a composition of the invention wherein the GR antagonist is selected from the group consisting of ORG 34517, 11-(substituted phenyl)-estra-4,9-diene derivatives, and 11-(substituted phenyl)-estra-4,9-diene derivatives of formula I

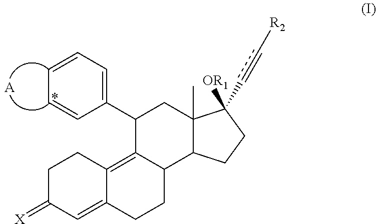

wherein A is a residue of a 5- or 6-membered ring containing 2 heteroatoms which are not connected to each other and independently selected from O and S, the ring being optionally substituted with one or more halogen atoms, or A is a residue of a 5- or 6-membered ring wherein no double C—C bonds are present, containing 1 heteroatom selected from O and S, which heteroatom is connected to the phenyl group at the position indicated with an asterisk, the ring being optionally substituted with one or more halogen atoms; R1 is H or 1-oxo(1-4C)alkyl; R2 is H, (1-8C)alkyl, halogen or CF3; X is selected from (H,OH), O, and NOH; and the interrupted line represents an optional bond, and combinations thereof.

The invention further provides a composition of the invention wherein the keratinous tissue is selected from the group consisting of toenails, fingernails, cuticles, claws, hooves, and combinations thereof.

The invention further provides a composition of the invention in a form selected from the group consisting of application stick, mascara, eyebrow coloring, eyeliner, eye shadow or other eye lid cosmetics, facial or body powder, sun tanning lotions and creams and sprays, sunscreens and blocks, nail polish, mousse, sprays, styling gels, nail conditioner, whether in the form of creams, lotions, gels, ointments, emulsions, colloids, solutions, suspensions, compacts, solids, pencils, brushes, spray-on formulations, brush-on formulations and the like, bath and shower gels, shampoos, conditioners, cream rinses, hair dyes and coloring products, leave-on conditioners, lip balms, skin conditioners, hair sprays, soaps, body scrubs, exfoliants, astringents, depilatories and permanent or temporary waving solutions, antidandruff formulations, antisweat and antiperspirant compositions, shaving, preshaving and after shaving products, moisturizers, deodorants, cold creams, cleansers, skin gels, rinses, whether in solid, powder, liquid, cream, gel, ointment, lotion, emulsions, colloids, solutions, suspensions, or other form, gels, patches, creams, sprays, ointments, lotions, emulsions, colloids, solutions, suspensions, powders and the like, and combinations thereof.

The invention further provides a composition of the invention further comprising at least one other ingredient selected from the group consisting of solvents, thickeners or gelling agents, coloring materials which are soluble in the medium of the composition, fillers, pigments, antioxidants, preservatives, fragrances, electrolytes, neutralizing agents, film-forming polymers, UV blocking agents, cosmetic and pharmaceutical active principles other than GR antagonists, and combinations thereof.

The invention further provides a composition of the invention wherein said at least one additional ingredient is a cleaning agent, hair conditioning agent, skin conditioning agent, hair styling agent, antidandruff agent, hair growth promoter, perfume, sunscreen, sunblock, pigment, moisturizer, film former, hair color, make-up agent, detergent, pharmaceutical, thickening agent, emulsifier, humectant, emollient, antiseptic agent, deodorant active, dermatologically acceptable carrier, surfactant, abrasive, absorbent, fragrance, coloring/colorant, essential oil, skin sensate, astringent, anti-acne agent, anti-caking agent, antifoaming agent, antimicrobial, antioxidant, binder, biological additive, enzyme, enzyme inhibitor, enzyme activator, coenzyme, botanical extract, ceramide, addition peptide, buffering agent, bulking agent, chelating agent, cosmetic biocide, denaturant drug astringent, external analgesic, polymer, quat, substantivity increasing agent, opacifying agent, pH adjuster, propellant, reducing agent, sequestrant, skin bleaching agent, skin lightening agent, skin-conditioning agent, skin soothing agent, skin healing agent, aloe vera, pantothenic acid and derivative thereof, allantoin, bisabolol, dipotassium glycyrrhizinate, skin treating agent, thickener, or a vitamin or derivative thereof and mixtures or combinations thereof.

The invention further provides a composition of the invention further comprising a plurality of additional ingredients and wherein at least one of said additional ingredients is a dermatologically acceptable carrier.

The invention provides a delivery device for the targeted application to the hair of a mammal a therapeutically effective amount of the composition of the invention.

The invention further provides a delivery device of the invention wherein the device is for the application of the composition to toenails, fingernails, cuticles, claws, hooves, and combinations thereof.

The invention provides a method for treating or preventing stress induced keratinous tissue damage wherein said method comprises administering, to a patient in need of such therapy, at least one GR antagonist in a therapeutically effective amount.

The invention further provides a method of the invention wherein the at least one GR antagonist is in a pharmaceutical preparation.

The invention further provides a method of the invention wherein the GR antagonist is selected from the group consisting of ORG 34517, 11-(substituted phenyl)-estra-4,9-diene derivatives, and 11-(substituted phenyl)-estra-4,9-diene derivatives of formula I

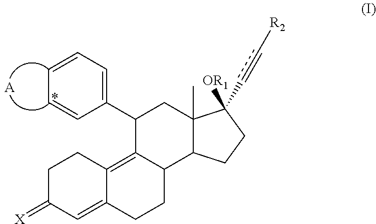

wherein A is a residue of a 5- or 6-membered ring containing 2 heteroatoms which are not connected to each other and independently selected from O and S, the ring being optionally substituted with one or more halogen atoms, or A is a residue of a 5- or 6-membered ring wherein no double C—C bonds are present, containing 1 heteroatom selected from O and S, which heteroatom is connected to the phenyl group at the position indicated with an asterisk, the ring being optionally substituted with one or more halogen atoms; R1 is H or 1-oxo(1-4C)alkyl; R2 is H, (1-8C)alkyl, halogen or CF3; X is selected from (H,OH), O, and NOH; and the interrupted line represents an optional bond.

The invention further provides a method of the invention wherein the hair is selected from the group consisting of toenails, fingernails, cuticles, claws, hooves, and combinations thereof.

The invention provides a method of treating or preventing keratinous tissue damage secondary to stress in a patient in need thereof, comprising administering at least one GR antagonist in a therapeutically effective amount.

The invention further provides a method of the invention wherein the stress is selected from the group consisting of chemotherapy, traumatic events, and combinations thereof.

The invention further provides a method of the invention wherein the hair is selected from the group consisting of toenails, fingernails, cuticles, claws, hooves, and combinations thereof.

The invention provides a kit for treating or preventing stress induced keratinous tissue damage comprising: (a) a pharmaceutical composition comprising at least one GR antagonist in a therapeutically effective amount; and (b) at least one blister package; a lidded blister; a blister card or packet; a clamshell; an intravenous (IV) package, IV packette or IV container; a tray or a shrink wrap comprising the pharmaceutical composition of (a) and instructions for use of the pharmaceutical composition for treating or preventing stress induced keratinous tissue damage.

The invention provides a product of manufacture for treating or preventing stress induced keratinous tissue damage comprising a blister package; a lidded blister; a blister card or packet; a clamshell; an intravenous (IV) package, IV packette or IV container; a tray or a shrink wrap comprising a pharmaceutical composition comprising at least one GR antagonist, and instructions for use of the pharmaceutical composition for treating or preventing stress induced keratinous tissue damage.

### DETAILED DESCRIPTION OF THE INVENTION

Telogen effluvium is sudden or severe stress related hair loss, which appears as thinning throughout the whole scalp.

A sudden or stressful event can cause the hair follicles to prematurely stop growing and enter into a resting phase. The hair will then stay in the resting phase for about 3 months after which time a large amount of hair will be shed. In most cases the hair loss is temporary and the hair soon recovers. However in some cases the hair loss continues until the underlying cause is removed.

Events which may lead to telogen effluvium include, for example, childbirth; termination of pregnancy; starting or stopping birth control pills; use of various medications; and severe emotional stress.

Increased levels of hormones estrogen and progesterone during pregnancy cause more hairs than normal to remain in the growth phase. Following childbirth or termination of pregnancy, many of the hair follicles that had delayed entering the resting phase suddenly enter the resting phase due to the rapid drop in hormone levels.

Birth control pills affect the hormone levels within the body and these hormone levels can affect hair growth. In some cases hair thinning may occur due to the male hormones present is some types of contraceptive pills, resulting in hair loss similar to that of androgenetic alopecia. Discontinuation of the pill can result in hair loss similar to that which occurs after childbirth, due to the drop in hormone levels.

Drugs which may cause hair loss as a side effect include anti-gout agents such as alloppurinol; blood thinners such as heparin and coumarin; and cholesterol lowering drugs such as clofibrate and gemfibrozil.

Telogen effluvium may also occur after a traumatic event such as the death of a loved one, an accident, abuse or any other severely traumatic event. These events may trigger hair follicles to enter the resting phase prematurely in which case an increase in the amount of hair shed will be noticed about 3 months after the event.

Other causes of telogen effluvium include thyroid gland malfunction (hypothyroidism or hyperthyroidism, which occurs when the thyroid gland produces too little or too much, respectively, of the thyroid hormone, thyroxin); diabetes; anaemia; and the autoimmune disease, systemic lupus erythematosis.

Scarring alopecia occurs as a result of inflammation of the hair follicles due to infection.

Scarring alopecia may be caused by discoid lupus erythematosus, a diffuse connective tissue disease; lichenplanus, which is an inflammatory disease that strikes primarily the skin and mucous membranes; Pseudopelade of Brocq, a rare scarring alopecia which has no potential for regrowth; aplasia cutis congenita, a rare disorder that often results as a small blistered atrophied area usually in the midline of the scalp and present from birth; or congenital ctrichia.

Other types of hair loss include syphilitic alopecia, a secondary manifestation of syphilis; scleroderma; tinea capitis (ringworm); hot spots; and flea or tick infestation.

In addition, chemotherapy frequently induces hair loss. With chemotherapy, patients not only experience reduced stamina and independence but also must wear a physical symbol of their illness in the loss of their hair. This loss of hair is a traumatic experience that may well result in lower self-esteem and overall resistance. Some patients are known to have refused chemotherapy for fear of losing their hair. Scalp tourniquets have been used for several decades to prevent chemotherapy-induced alopecia. This technique involves the placement of a pneumatic tourniquet around the hairline at the time of administration of the chemotherapeutic drug. The tourniquet is then inflated to a pressure above the systolic arterial pressure, reducing blood flow to the scalp. The effectiveness of this technique has never been unambiguously demonstrated. The use of tourniquets has more or less been replaced by scalp hypothermia. With this technique, the scalp temperature is lowered to below 24° C. by application of cold packs, etc., prior to chemotherapy. The technique has been reported to afford a 50-70% good to excellent hair protective effect. However, results have remained notoriously variable. Furthermore, the practice is rather uncomfortable and is only tolerated for a short time. It is likely to be most effective for chemotherapy agents with short half lives. Moreover, several cases of scalp metastases in patients who used scalp hypothermia were reported. Finally, the technique appears to work considerably less well for combination chemotherapy than for therapy using single agents. Several pharmacological approaches for the prevention of chemotherapy-induced hair loss were also tested. For a review, see Dorr. 1998. Semin. Oncol. 25: 562-570. Most of the drugs tested failed (for example, alpha-tocopherol, minoxidil, calcitriol) or showed a marked sex preference (1,25-dihydroxy vitamin D3). There is a need for additional drug candidates and methods for protection against chemotherapy-induced alopecia.

Hence, while the prior art teaches the use of various medications for treating the different types of alopecia, these treatments are oftentimes limited by high cost, prolonged treatment periods, insufficient efficacy and/or adverse side effects.

There is thus a widely recognized need for, and it would be highly advantageous to have, novel compositions and methods for treating one or more of the above-cited alopecia types, devoid of the above limitations.

The present disclosure relates to methods for treating or preventing stress induced hair loss wherein said method comprises administering, to a patient in need of such therapy, at least one GR antagonist in a therapeutically effective amount. The present disclosure also relates to composition, including for example, topical composition for treating or preventing stress induced hair loss in a patient comprising a hair growth stimulating agent which is at least one GR antagonist in a therapeutically effective amount, along with cosmetically or pharmaceutically acceptable excipient, carrier or vehicle, wherein the composition has the effect of treating or preventing stress induced hair loss in the patient.

An amount is “effective” as used herein, when the amount provides an effect in promoting hair growth in the subject. As used herein, the term “effective amount” means an amount of a compound or composition sufficient to significantly induce a positive benefit, such as a positive keratinous tissue appearance or feel benefit, including independently or in combinations the benefits disclosed herein, but low enough to avoid serious side effects, i.e., to provide a reasonable benefit to risk ratio, within the scope of sound judgment of the skilled artisan. For those skilled in the art, the effective amount, as well as dosage and frequency of administration, may easily be determined according to their knowledge and standard methodology of merely routine experimentation based on the present disclosure.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

### Hair

As used herein the term “hair” is meant to encompass all mammalian hair. While in humans the hair whose growth is to be regulated is typically located upon the head, scalp, eyebrows, eyelids, eyelashes, mustaches, beards, chest, arms, legs, and the pubic area, the inventive method and compositions described herein may be applied to hair located anywhere on the body.

As used herein, the terms “hair growth,” “regulating hair growth,” “hair growth regulation,” and “regulating the growth of hair,” are meant to include: stimulating hair growth; stimulating hair thickening; preventing, reducing, arresting and/or retarding the loss of hair; preventing, reducing, arresting and/or retarding the thinning of hair; increasing the rate of hair growth; inducing the formation of a greater number of hair strands; increasing the diameter of the hair strand; lengthening the hair strand; changing the hair follicle from vellus follicle to terminal follicle; inducing the formation of vellus follicles.

The term “hair follicle” as used herein refers to a hair structure underneath the skin with a stocking-like structure that contains several layers with different functions. Each hair passes through three distinct phases, namely anagen, catagen and telogen between starting to grow and falling out years later. The “anagen” phase is the active growth phase of hair follicles. The cells in the root of the hair are dividing rapidly, adding to the hair shaft. The “catagen” phase refers to a short transition phase that occurs at the end of the anagen phase. It signals the end of the active growth of a hair. The “telogen” phase is the resting phase of the hair follicle. During this phase the hair follicle is completely at rest and the club hair (where the hair root is surrounded by a bulbous enlargement composed of completely keratinized cells) is completely formed.

The term “keratinous tissue,” as used herein, refers to keratin-containing layers disposed as the outermost protective covering of mammals (e.g., humans, dogs, cats, etc.) that includes, but is not limited to, skin, lips, hair, toenails, fingernails, cuticles, claws, hooves, etc.

The term “topical application”, as used herein, means to apply or spread the compositions of the present invention onto the surface of the keratinous tissue.

The term “dermatologically-acceptable,” as used herein, means that the described compositions or components thereof are suitable for use in contact with mammalian keratinous tissue without undue toxicity, incompatibility, instability, allergic response, and the like. As used herein, therapeutically regulating keratinous tissue condition includes ameliorating, e.g., diminishing, minimizing and/or effacing, damge, such as, discontinuities in keratinous tissue (e.g., skin, hair, or nails). This is also encompassed within the term “treating.”

The present invention relates to a method for improving or regulating keratinous tissue condition. In one aspect, this may be accomplished by providing the composition described herein and applying the composition to keratinous tissue in need of treatment, for example, keratinous tissue which is damaged. Conditions to be improved or regulated include thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin; change in coloration to the skin, hair, or nails, for example, under-eye circles, blotchiness (e.g., uneven red coloration due to, for example, rosacea), sallowness, discoloration caused by telangiectasia or spider vessels, dryness, brittleness, and graying hair.

As used herein, prophylactically regulating keratinous tissue damage condition includes delaying, minimizing and/or preventing visible and/or tactile discontinuities in keratinous tissue (e.g., texture irregularities in the skin, hair, or nails which may be detected visually or by feel), including signs of skin, hair, or nail aging. This is also encompassed within the term “treating.”

### Cosmetics

“Cosmetics,” as used herein, include without limitation, application stick, mascara, eyebrow coloring, eyeliner, eye shadow or other eye lid cosmetics, facial or body powder, sun tanning lotions and creams and sprays, sunscreens and blocks, nail polish, mousse, sprays, styling gels, nail conditioner, whether in the form of creams, lotions, gels, ointments, emulsions, colloids, solutions, suspensions, compacts, solids, pencils, brushes, spray-on formulations, brush-on formulations and the like.

### Personal Care Products

“Personal care products” include, without limitation, bath and shower gels, shampoos, conditioners, cream rinses, hair dyes and coloring products, leave-on conditioners, sun tanning lotions and creams and sprays, sunscreens and sunblocks, lip balms, skin conditioners, hair sprays, soaps, body scrubs, exfoliants, astringents, depilatories and permanent or temporary waving solutions, antidandruff formulations, antisweat and antiperspirant compositions, shaving, preshaving and after shaving products, moisturizers, deodorants, cold creams, cleansers, skin gels, rinses, whether in solid, powder, liquid, cream, gel, ointment, lotion, emulsions, colloids, solutions, suspensions, magnesium based bath salts, or other form.

### Glucocorticoid Receptor Antagonists

GR antagonists bind to the receptor and prevent GR agonists from binding and eliciting GR mediated events, including transcription. RU486 is an example of a non-selective GR antagonist.

Compounds having high GR binding affinity and, in addition, high in vivo anti-glucocorticoid activity, while having, for example, low androgenic and progestagenic activities are disclosed in U.S. Pat. No. 6,011,025, incorporated herein by reference in its entirety. ORG 34517 is an example of a compound with high GR binding affinity while having low androgenic and progestagenic activities.

It has been found that 11-(substituted phenyl)-estra-4,9-diene derivatives of formula I

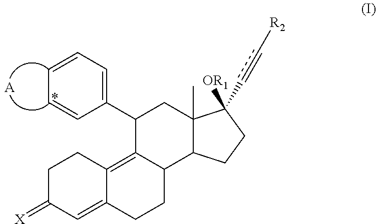

wherein
A is a residue of a 5- or 6-membered ring containing 2 heteroatoms which are not connected to each other and independently selected from O and S, the ring being optionally substituted with one or more halogen atoms, or A is a residue of a 5- or 6-membered ring wherein no double C—C bonds are present, containing 1 heteroatom selected from O and S, which heteroatom is connected to the phenyl group at the position indicated with an asterisk, the ring being optionally substituted with one or more halogen atoms; R1 is H or 1-oxo(1-4C)alkyl; R2 is H, (1-8C)alkyl, halogen or CF3; X is selected from (H,OH), O, and NOH; and the interrupted line represents an optional bond, show specific and high GR binding affinity and are highly active in vivo showing predominant anti-glucocorticoid activity.

The compounds lack appreciable affinity for mineralocorticoid, progesterone, estrogen and androgen receptors, indicating a clean side effect profile.

The 11-(substituted phenyl)-estra-4,9-diene derivatives of the invention can be used in the prevention and treatment of glucocorticoid dependent diseases or symptoms, like Cushing syndrome, diabetes, glaucoma, sleep disturbances, depression, anxiety, atherosclerosis, hypertension, adiposity, osteoporosis and withdrawal symptoms from narcotics and their mixtures.

Preferred compounds according to this invention are 11-(substituted phenyl) estra-4,9-diene derivatives, wherein the heteroatom(s) are (is) O, the 5- or 6-membered ring being optionally substituted with one or more fluorine atoms; R1 is H; and X is O or NOH. More preferred compounds are 11-(substituted phenyl) estra-4,9-diene derivatives wherein A is a residue of a 5-membered ring. Particularly preferred are 11-(substituted phenyl) estra-4,9-diene derivatives wherein A contains 2 heteroatoms being O.

Especially preferred are 11-(substituted phenyl) estra-4,9-diene derivatives wherein R2 is methyl and the interrupted line represents a bond.

The most preferred compound is (11β,17β-11-(1,3-benzodioxol-5-yl)-17-hydroxy-17-(1-propynyl) estra-4,9-dien-3-one (ORG 34517).

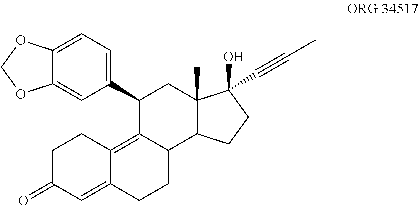

The term halogen means a fluorine, chlorine, bromine or iodine atom. Fluorine is the preferred halogen in ring A and when R2 is halogen, chlorine is preferred.

The terms (1-4C)alkyl and (1-8C)alkyl, as used in the definitions of R1 and R2, respectively, mean alkyl groups having 1-4 and 1-8 carbon atoms, respectively, for example methyl, ethyl, propyl, isopropyl, butyl, sec-butyl, tert-butyl, pentyl, neopentyl, hexyl, octyl.

The 11-(substituted phenyl)-estra-4,9-diene derivatives according to the present invention can be prepared by a process wherein a compound of formula II

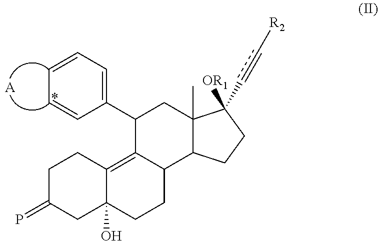

wherein A, R2 and the interrupted line have the meanings as previously defined, R1 is H, and P is a protected keto-group, is dehydrated and deprotected, after which the 17β-OH is optionally esterified by reaction with an appropriate carboxylic acid to give a derivative wherein R1 is 1-oxo(1-4C)alkyl, and optionally the 3-oxo group is converted into the corresponding 3-hydroxy- or 3-oxime derivative. The 3-oxo group can be reduced to form the 3-hydroxy-derivative by using a suitable reducing agent, such as sodium borohydride. The 3-oxime derivatives can be prepared by hydroxylamine treatment in a suitable solvent, like pyridine.

The derivatives of formula II may be prepared according to well known methods described and used for the preparation of steroids.

A suitable process for the preparation of derivatives of formula II starts from estra-4,9-diene-3,17-dione. Selective reduction of the 17-keto group to 17β-OH, 17α-H, e.g. with sodium borohydride, followed by protection of the 3-keto group, e.g., by ketalisation with ethyleneglycol, triethylorthoformate and p-toluenesulfonic acid, and oxidation of the 17-hydroxy group, e.g., with pyridinium chlorochromate, provides the 3-ketoprotected estra-5(10),9(11)-diene-3,17-dione. Alkynylation at the 17-position (yielding a 17α-alkynyl,17β-OH derivative), followed by epoxidation of the 5(10) double bond, e.g., with hydrogen peroxide, trifluoroacetophenone, and pyridine in dichloromethane according to the method as disclosed in European patent application EP 0 298 020, provides the 3-ketoprotected 5α,10α-epoxy-17α-alkynyl-17β-hydroxy-estr-9(11)-ene-3-one.

Subsequently, compounds of formula II are formed from this epoxide derivative, for example by reaction with an organometallic compound of the formula

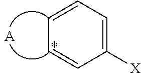

wherein X is a (alkali)metal, like lithium, or a magnesiumhalide, preferably magnesium bromide.

Suitable protective groups and methods to remove these groups are known in the art, for example from T. W. Green: Protective Groups in Organic Synthesis (Wiley, NY, 1981). Particularly suitable protective groups for the protection of keto groups are acetals, e.g., 1,2-ethylene ketal.

The specificity of ORG 34517 for GR blockade, without significant cross-binding to other related steroidal hormone receptors (such as those for estrogen and progesterone), eliminates the likelihood of significant toxicities and side effects. Indeed, none were identified in all the substantial phase I and phase II clinical trials that already have been performed with the compound. Because the drug is envisioned as being used in limited dosing over time, coordinated with the intermittent dosing strategies typical for chemotherapeutic agents, the GR blockade also would not lead to significant alteration of HPA-axis functioning, with rapid restitution of the HPA-axis to baseline following dosing.

### Formulations

The compounds of the invention may be, for example, administered locally. The compounds of the invention may be, for example, administered topically. The compounds of the invention may be, for example, mixed with pharmaceutically suitable auxiliaries, e.g., as described in the standard reference, Gennaro et al., Remington's Pharmaceutical Sciences. By means of pharmaceutically suitable liquids the compounds may be applied in the form of, for example, a solution, suspension, or emulsion. The compounds may also be formulated in, for example, a patch, ointment or can be enclosed in a device for local administration to the skin.

The compounds of the invention may be administered enterally or parenterally. Mixed with pharmaceutically suitable auxiliaries, e.g., as described in the standard reference, Gennaro et al., Remington's Pharmaceutical Sciences, 2005. The compounds may be compressed into solid dosage units, such as pills, tablets, or be processed into capsules or suppositories. By means of pharmaceutically suitable liquids the compounds can also be applied in the form of a solution, suspension, emulsion, e.g., for use as an injection preparation or eye drops, or as a spray, e.g., for use as a nasal spray.

For making dosage units, e.g., tablets, the use of conventional additives such as fillers, colorants, polymeric binders and the like is contemplated. In general, any pharmaceutically acceptable additive which does not interfere with the function of the active compounds can be used. Suitable carriers with which the compositions can be administered include lactose, starch, cellulose derivatives and the like, or mixtures thereof, used in suitable amounts.

### Dosage Forms

The compositions of the present invention can be processed by agglomeration, air suspension chilling, air suspension drying, balling, coacervation, coating, comminution, compression, cryopelletization, encapsulation, extrusion, wet granulation, dry granulation, homogenization, inclusion complexation, lyophilization, melting, microencapsulation, mixing, molding, pan coating, solvent dehydration, sonication, spheronization, spray chilling, spray congealing, spray drying, or other processes known in the art. The compositions can be provided in the form of a minicapsule, a capsule, a tablet, an implant, a troche, a lozenge (minitablet), a temporary or permanent suspension, an ovule, a suppository, a wafer, a chewable tablet, a quick or fast dissolving tablet, an effervescent tablet, a buccal or sublingual solid, a granule, a film, a sprinkle, a pellet, a bead, a pill, a powder, a triturate, a platelet, a strip or a sachet. Compositions can also be administered as a “dry syrup”, where the finished dosage form is placed directly on the tongue and swallowed or followed with a drink or beverage. These forms are well known in the art and are packaged appropriately. The compositions can be formulated for oral, nasal, buccal, ocular, urethral, transmucosal, vaginal, topical or rectal delivery.

The pharmaceutical composition can be coated with one or more enteric coatings, seal coatings, film coatings, barrier coatings, compress coatings, fast disintegrating coatings, or enzyme degradable coatings. Multiple coatings can be applied for desired performance. Further, the dosage form can be designed for immediate release, pulsatile release, controlled release, extended release, delayed release, targeted release, synchronized release, or targeted delayed release. For release/absorption control, solid carriers can be made of various component types and levels or thicknesses of coats, with or without an active ingredient. Such diverse solid carriers can be blended in a dosage form to achieve a desired performance. The definitions of these terms are known to those skilled in the art. In addition, the dosage form release profile can be affected by a polymeric matrix composition, a coated matrix composition, a multiparticulate composition, a coated multiparticulate composition, an ion-exchange resin-based composition, an osmosis-based composition, or a biodegradable polymeric composition. Without wishing to be bound by theory, it is believed that the release may be effected through favorable diffusion, dissolution, erosion, ion-exchange, osmosis or combinations thereof.

When formulated as a capsule, the capsule can be a hard or soft gelatin capsule, a starch capsule, or a cellulosic capsule. Although not limited to capsules, such dosage forms can further be coated with, for example, a seal coating, an enteric coating, an extended release coating, or a targeted delayed release coating. These various coatings are known in the art, but for clarity, the following brief descriptions are provided: seal coating, or coating with isolation layers: Thin layers of up to 20 microns in thickness can be applied for variety of reasons, including for particle porosity reduction, to reduce dust, for chemical protection, to mask taste, to reduce odor, to minimize gastrointestinal irritation, etc. The isolating effect is proportional to the thickness of the coating. Water soluble cellulose ethers are preferred for this application. HPMC and ethyl cellulose in combination, or Eudragit E 100, may be particularly suitable for taste masking applications. Traditional enteric coating materials listed elsewhere can also be applied to form an isolating layer.

Extended release coatings are designed to effect delivery over an extended period of time. The extended release coating is a pH-independent coating formed of, for example, ethyl cellulose, hydroxypropyl cellulose, methylcellulose, hydroxymethyl cellulose, hydroxyethyl cellulose, acrylic esters, or sodium carboxymethyl cellulose. Various extended release dosage forms can be readily designed by one skilled in art to achieve delivery to both the small and large intestines, to only the small intestine, or to only the large intestine, depending upon the choice of coating materials and/or coating thickness.

Enteric coatings are mixtures of pharmaceutically acceptable excipients which are applied to, combined with, mixed with or otherwise added to the carrier or composition. The coating may be applied to a compressed or molded or extruded tablet, a gelatin capsule, and/or pellets, beads, granules or particles of the carrier or composition. The coating may be applied through an aqueous dispersion or after dissolving in appropriate solvent. Additional additives and their levels, and selection of a primary coating material or materials will depend on the following properties: 1. resistance to dissolution and disintegration in the stomach; 2. impermeability to gastric fluids and drug/carrier/enzyme while in the stomach; 3. ability to dissolve or disintegrate rapidly at the target intestine site; 4. physical and chemical stability during storage; 5. non-toxicity; 6. easy application as a coating (substrate friendly); and 7. economical practicality.

Dosage forms of the compositions of the present invention can also be formulated as enteric coated delayed release oral dosage forms, i.e., as an oral dosage form of a pharmaceutical composition as described herein which utilizes an enteric coating to affect release in the lower gastrointestinal tract. The enteric coated dosage form may be a compressed or molded or extruded tablet/mold (coated or uncoated) containing granules, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

Delayed release generally refers to the delivery so that the release can be accomplished at some generally predictable location in the lower intestinal tract more distal to that which would have been accomplished if there had been no delayed release alterations. The preferred method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention to achieve delivery to the lower gastrointestinal tract. Polymers for use in the present invention are anionic carboxylic polymers.

Shellac, also called purified lac, a refined product obtained from the, resinous secretion of an insect. This coating dissolves in media of pH>7.

Colorants, detackifiers, surfactants, antifoaming agents, lubricants, stabilizers such as hydroxy propyl cellulose, acid/base may be added to the coatings besides plasticizers to solubilize or disperse the coating material, and to improve coating performance and the coated product.

In carrying out the method of the present invention, the combination of the invention may be administered to mammalian species, such as dogs, cats, humans, etc. and as such may be incorporated in a conventional systemic dosage form, such as a tablet, capsule, elixir or injectable. The above dosage forms will also include the necessary carrier material, excipient, lubricant, buffer, antibacterial, bulking agent (such as mannitol), anti-oxidants (ascorbic acid of sodium bisulfite) or the like.

The dose administered must be carefully adjusted according to age, weight and condition of the patient, as well as the route of administration, dosage form and regimen and the desired result.

The pharmaceutical compositions of the invention may be administered in the dosage forms in single or divided doses of one to four times daily. It may be advisable to start a patient on a low dose combination and work up gradually to a high dose combination.

Tablets of various sizes can be prepared, e.g., of about 1 to 2000 mg in total weight, containing one or both of the active pharmaceutical ingredients, with the remainder being a physiologically acceptable carrier of other materials according to accepted pharmaceutical practice. These tablets can be scored to provide for fractional doses. Gelatin capsules can be similarly formulated.

Liquid formulations can also be prepared by dissolving or suspending one or the combination of active substances in a conventional liquid vehicle acceptable for pharmaceutical administration so as to provide the desired dosage in one to four teaspoonful.

Dosage forms can be administered to the patient on a regimen of, for example, one, two, three, four, five, six, or other doses per day

In order to more finely regulate the dosage schedule, the active substances may be administered separately in individual dosage units at the same time or carefully coordinated times. Since blood levels are built up and maintained by a regulated schedule of administration, the same result is achieved by the simultaneous presence of the two substances. The respective substances can be individually formulated in separate unit dosage forms in a manner similar to that described above.

In formulating the compositions, the active substances, in the amounts described above, may be compounded according to accepted pharmaceutical practice with a physiologically acceptable vehicle, carrier, excipient, binder, preservative, stabilizer, flavor, etc., in the particular type of unit dosage form.

Illustrative of the adjuvants which may be incorporated in tablets are the following: a binder such as gum tragacanth, acacia, corn starch or gelatin; an excipient such as dicalcium phosphate or cellulose; a disintegrating agent such as corn starch, potato starch, alginic acid or the like; a lubricant such as stearic acid or magnesium stearate; a sweetening agent such as sucrose, aspartame, lactose or saccharin; a flavoring agent such as orange, peppermint, oil of wintergreen or cherry. When the dosage unit form is a capsule, it may contain in addition to materials of the above type a liquid carrier such as a fatty oil. Various other materials may be present as coatings or to otherwise modify the physical form of the dosage unit. For instance, tablets or capsules may be coated with shellac, sugar or both. A syrup of elixir may contain the active compound, water, alcohol or the like as the carrier, glycerol as solubilizer, sucrose as sweetening agent, methyl and propyl parabens as preservatives, a dye and a flavoring such as cherry or orange.

One embodiment of this invention includes methods of treating, preventing, or diagnosing a particular disease or condition by administering the disclosed nanoparticles, composite nanoparticles, nanosuspension, or nanocapsules to a subject. In many instances, the nanoparticles, composite nanoparticles, or nanocapsules are administered alone or can be included within a pharmaceutical composition. An effective amount of a pharmaceutical composition, generally, is defined as that amount sufficient to ameliorate, reduce, minimize, or limit the extent of the disease or condition. More rigorous definitions may apply, including elimination, eradication, or cure of the disease or condition.

“Nanoparticles” are solid particles of an average particle diameter of, for example, less than about 1 micron (micrometer). One micron is 1,000 nanometers (nm).

“Stabilized” nanoparticles are nanoparticles coated with a stabilizing material and having a reduced tendency for aggregation and loss of dispersion with respect to nanoparticles of the compound of the invention without a stabilizing coating.

A nano-spray is a spray containing nanoparticles or a spray that produces nanoparticles. A nanodispersion is a dispersion containing nanoparticles. A nanosuspension is a suspension containing nanoparticles.

The liquid formulations useful herein may comprise a solvent, solution, suspension, microsuspension, nanosuspension, emulsion, microemulsion, gel or even a melt containing the active component or components. In some embodiments the nanoparticles, nanofibers, or nanofibrils may be in the form of, or within or on, granules, powders, suspensions, solutions, dissolvable films, mats, webs, tablets, or releasable forms particularly releasable dosage forms. Other particular useful forms are concentrates to which a diluting liquid is added prior to use. The product may also be sprayed onto the inner surface of a container to which a liquid is added later prior to use and the nanoparticles, nanofibers, or nanofibrils, are released into the liquid.

Pharmaceutical compositions of the present invention can include nanoparticles, composite nanoparticles, nanosuspension, or nanocapsules of the present invention.

In certain non-limiting embodiments, pharmaceutical compositions may comprise, for example, at least about 0.1% of an active ingredient or nanoparticles, composite nanoparticles, or nanocapsules, for example. In other embodiments, the an active ingredient or nanoparticles, composite nanoparticles, or nanocapsules may comprise between about 2% to about 75% of the weight of the unit, or between about 25% to about 60%, for example, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered.

The composition may also include various antioxidants to retard oxidation of one or more active ingredient or nanoparticles, composite nanoparticles, nanosuspension, or nanocapsules. The prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In order to increase the effectiveness of a treatment with the nanoparticles, nanogels, composite nanoparticles, nanosuspension, or nanocapsules of the present invention, it may be desirable to combine these nanoparticles, composite nanoparticles, or nanocapsules with other therapies effective in the treatment of a particular disease or condition.

The formulations as described above may be administered for a prolonged period, that is, for as long as the potential for a disease or condition remains or the symptoms continue.

### Topical Formulations

The term “topical” as employed herein relates to the use of a compound, derivative or analogue as described herein, incorporated in a suitable pharmaceutical carrier, and applied at the site of thinning hair for exertion of local action. Accordingly, such topical compositions including those forms in which the compound is applied externally by direct contact with the skin surface to be treated. Conventional forms for this purpose include ointments, liniments, creams, shampoos, lotions, pastes, jellies, sprays, aerosols, soaps, and the like, and may be applied in patches or impregnated dressings depending on the part of the body to be treated. The term “ointment” embraces formulations (including creams) having oleaginous, absorption, water-soluble and emulsion-type bases, e.g., petrolatum, lanolin, polyethylene glycols, as well as mixtures of these.

For topical use on the skin, eyelids, eyebrows, the active hair growth stimulating agents as well as their derivatives and analogues, including esters and salts, can be formulated in aqueous solutions, creams, ointments or oils exhibiting physiologically acceptable osmolarity by addition of pharmacologically acceptable buffers and salts. Such formulations may or may not, depending on the dispenser, contain preservatives such as benzalkonium chloride, chlorhexidine, chlorobutanol, parahydroxybenzoic acids and phenylmercuric salts such as nitrate, chloride, acetate, and borate, or antioxidants, as well as additives like EDTA, sorbitol, boric acid etc. as additives. Furthermore, particularly aqueous solutions may contain viscosity increasing agents such as polysaccharides, e.g., methylcellulose, mucopolysaccharides, e.g., hyaluronic acid and chondroitin sulfate, or polyalcohol, e.g., polyvinylalcohol. Various slow releasing gels and matrices may also be employed as well as soluble and insoluble ocular inserts, for instance, based on substances forming in-situ gels. Depending on the actual formulation and the hair growth stimulating agents to be used, various amounts of the drug and different dose regimens may be employed. Typically, the daily amount of the hair growth stimulating and/or hair loss prevention agent may be about 0.1 ng to about 100 mg, or about 1 ng to about 100 μg, but may be out of that range.

For topical use, the agent of the invention can be advantageously formulated using ointments, creams, liniments or patches as a carrier of the active ingredient. Also, these formulations may or may not contain preservatives, depending on the dispenser and nature of use. Such preservatives include those mentioned above, and methyl-, propyl-, or butyl-parahydroxybenzoic acid, betain, chlorhexidine, benzalkonium chloride, and the like. Various matrices for slow release delivery may also be used. Typically, the dose to be applied is in the range of about 0.1 ng to about 100 mg per day, or about 1 ng to about 10 mg per day, or about 10 ng to about 1 mg per day depending on the hair growth stimulating agent and the formulation. To achieve the daily amount of medication depending on the formulation, the hair growth stimulating and/or hair loss prevention agent may be administered once or several times daily, with or without antioxidants.

The composition or regime for application can be provided in the form of a hair care lotion, at various times, including but not limited to for example for daily or twice-weekly application, of a shampoo or of a hair conditioner, in particular for twice-weekly or weekly application, of a liquid or solid soap for cleaning, for daily application, of a product for shaping the hairstyle (lacquer, hairsetting product, styling gel), of a treatment mask, of a cream or of a foaming gel for cleaning the hair. It can also be provided in the form of a hair dye or mascara to be applied with a brush or comb.

Furthermore, for application to the eyelashes or body hairs, the composition and regime to which the invention relates can be provided in the form of a pigmented or nonpigmented mascara, applied with a brush pencil or other applicator device to the eyelashes or alternatively to the beard or moustache hairs, or scalp.

According to a specific embodiment, the composition and regime according to the invention is provided in the form of a hair cream or lotion, shampoo, hair conditioner, hair mascara, or other hair coloring device, or of a mascara eyeliner, or pencil.

Non-limiting examples of topical products can include, without limitation, application stick, mascara, eyebrow coloring products, eye shadow or other eye lid coloring products, eyeliner, make-up removal products, antiaging products, facial or body powder, nail polish, mousse, sprays, styling gels, nail conditioner, bath and shower gels, shampoos, conditioners, cream rinses, hair dyes and coloring products, hair conditioners, sun tanning lotions and creams and sprays, sunscreens and sunblocks, skin conditioners, cold creams, moisturizers, hair sprays, soaps, body scrubs, exfoliants, astringents, depilatories and permanent waving solutions, antidandruff formulations, antisweat and antiperspirant compositions, shaving, preshaving and after shaving products, moisturizers, deodorants, cold creams, cleansers, skin gels, and rinses.

Furthermore, the topical product can be applied topically through the use of a patch or other delivery device. Delivery devices can include, but are not limited to, those that can be heated or cooled, as well as those that utilize iontophoresis or ultrasound.

For instance, the topical product can be applied, for example, by applying a composition in the form of a skin lotion, clear lotion, milky lotion, cream, gel, foam, ointment, paste, emulsion, spray, conditioner, tonic, cosmetic, application stick, pencil, foundation, nail polish, after-shave, or the like which is intended to be left on the skin or other keratinous tissue (i.e., a “leave-on” composition). After applying the composition to the keratinous tissue (e.g., skin), it in one embodiment, it is left on for a period of at least about 15 minutes, or at least about 30 minutes, or at least about 1 hour, or for at least several hours, e.g., up to about 12 hours. In one embodiment, the topical product is left on overnight. In another embodiment, the topical product is left on all day. Any part of the external portion of the face, hair, and/or nails can be treated, (e.g., face, lips, under-eye area, eyelids, scalp, neck, torso, arms, legs, chest, hands, legs, feet, fingernails, toenails, scalp hair, eyelashes, eyebrows, etc.)

Any suitable method can be used to apply the topical product, including but not limited to for example using the palms of the hands and/or fingers or a device or implement (e.g., a cotton ball, swab, pad, applicator pen, spray applicator, eyebrow brush, eyebrow brush pencil, pencil, mascara brush, etc.) Another approach to ensure a continuous exposure of the keratinous tissue to at least a minimum level of the topical product is to apply the compound by use of a patch applied, e.g., to the face. The patch can be occlusive, semi-occlusive or non-occlusive, and can be adhesive or non-adhesive. The topical product can be contained within the patch or be applied to the skin prior to application of the patch. The patch can also include additional actives such as chemical initiators for exothermic reactions such as those described in PCT application WO 9701313, and in U.S. Pat. Nos. 5,821,250, 5,981,547, and 5,972,957 to Wu, et al. The patch can be left on the for any suitable period of time. For example, a period of at least about 5 minutes, or at least about 15 minutes, or at least about 30 minutes, or at least about 1 hour, or at night as a form of night therapy, or in another embodiment all day.

The topical product can comprise any suitable desired materials. For instance, such materials can be selected from the group consisting of sugar amines (e.g., N-acetylglucosamine), vitamin B3 compounds, sodium dehydroacetate, dehydroacetic acid and its salts, phytosterols, soy derivaties (e.g., equol and other isoflavones), niacinamide, phytantriol, farnesol, bisabolol, salicylic acid compounds, hexamidines, dialkanoyl hydroxyproline compounds, flavonoids, N-acyl amino acid compounds, retinoids (e.g., retinyl propionate), water-soluble vitamins, ascorbates (e.g., vitamin C, ascorbic acid, ascorbyl glucoside, ascorbyl palmitate, magnesium ascorbyl phosphate, sodium ascorbyl phosphate), particulate materials, sunscreen actives, anti-cellulite agents, butylated hydroxytoluene, butylated hydroxyanisole, their derivatives, and combinations thereof. Other examples include cationic polymers, conditioning agents (hydrocarbon oils, fatty esters, silicones), anti-dandruff agents, suspending agents, viscosity modifiers, dyes, nonvolatile solvents or diluents (water soluble and insoluble), pearlescent aids, foam boosters, surfactants, nonionic cosurfactants, pediculocides, pH adjusting agents, perfumes, preservatives, chelants, chelating agents, proteins, UV absorbers, pigments, other amino acids, and other vitamins.

For instance, topical products for use herein may comprise one or more vitamins and/or amino acids such as: water soluble vitamins such as vitamin B1, B2, B6, B12, C, pantothenic acid, pantothenyl ethyl ether, panthenol, biotin, and their derivatives, water soluble amino acids such as asparagine, alanine, indole, glutamic acid and their salts, water insoluble vitamins such as vitamin A, D, E, and their derivatives, water insoluble amino acids such as tyrosine, tryptamine, and their salts.

Topical products may also contain one or more pigment materials such as inorganic, nitroso, monoazo, disazo, carotenoid, triphenyl methane, triaryl methane, xanthene, quinoline, oxazine, azine, anthraquinone, indigoid, thionindigoid, quinacridone, phthalocianine, botanical, natural colors, including: water soluble components such as those having C. I. Names. The topical products may also contain antimicrobial agents which are useful as cosmetic biocides and antidandruff agents including: water soluble components such as piroctone olamine, water insoluble components such as 3,4,4′-trichlorocarbanilide (trichlosan), triclocarban and zinc pyrithione.

In some embodiments, the composition is chronically applied to the skin. By “chronic topical application” is meant continued topical application of the composition over an extended period during the subject's lifetime, for example, for a period of at least about one week, or for a period of at least about one month, or for at least about three months, or for at least about six months, or for at least about one year. While benefits are obtainable after various periods of use (e.g., five, ten or twenty years), chronic application can continue throughout the subject's lifetime. Typically applications would be on the order of about once per day over such extended periods, however application rates can vary from about once per week up to about three times per day or more.

A wide range of quantities of the compositions of the present invention can be employed to provide a skin appearance and/or feel benefit. Quantities of the present compositions that are typically applied per application are, in mg composition/cm² skin, from about 0.01 mg/cm² to about 10 mg/cm². A particularly useful application amount is about 1 mg/cm² to about 2 mg/cm².

Regulating keratinous tissue condition can be practiced by applying a composition of the invention in the form of a skin lotion, cream, gel, foam, ointment, paste, emulsion, spray, conditioner, tonic, cosmetic, lipstick, foundation, nail polish, after-shave, or the like that is preferably intended to be left on the skin or other keratin structure for some esthetic, prophylactic, therapeutic or other benefit (i.e., a “leave-on” composition). After applying the composition to the skin, it can be left on the skin for a period of at least about 15 minutes, or at least about 30 minutes, or at least about 1 hour, or for at least several hours, for example, up to about 12 hours.

Any part of the external portion of the face, hair, and/or nails can be treated, e.g., face, lips, under-eye area, eyelids, scalp, neck, torso, arms, hands, legs, feet, fingernails, toenails, scalp hair, eyelashes, eyebrows, etc. The composition can be applied with the fingers or with an implement or device (e.g., pad, cotton ball, applicator pen, spray applicator, and the like).

### Additional Ingredients

In addition to the agent of the invention, the compositions of the invention may include various other and additional ingredients, which may be active, functional, conventionally used in cosmetic, personal care or topical/transdermal pharmaceutical products or otherwise. Of course, a decision to include an additional ingredient and the choice of specific additional ingredients depends on the specific application and product formulation. Also, the line of demarcation between an “active” ingredient and an “inactive ingredient” is artificial and dependent on the specific application and product type. A substance that is an “active” ingredient in one application or product may be a “functional” ingredient in another, and vice versa. A particular ingredient might provide substantivity in one formulation, facilitate transdermal application in another, and merely provide proper viscosity in a third. Which of these is functional and which is active is subject to debate. But, regardless of the outcome, the material in question would qualify as an additional ingredient in accordance with the present invention.

Thus, the compositions of the invention may include one or more additional ingredients, which provide some benefit to the object of the composition. Such additional ingredients may include one or more substances such as, without limitations, cleaning agents, hair conditioning agents, skin conditioning agents, hair styling agents, antidandruff agents, hair growth promoters, perfumes, sunscreen and/or sunblock compounds, pigments, moisturizers, film formers, hair colors, make-up agents, detergents, pharmaceuticals, thickening agents, emulsifiers, humectants, emollients, antiseptic agents, deodorant actives, dermatologically acceptable carriers, surfactants, hair straightening agents, hair coloring agents, hair bleaching agents, and combinations thereof.

The compositions of the present invention generally contain at least one additional ingredient. The compositions of the present invention may contain a plurality of additional ingredients as well.

Where the composition is to be in contact with human keratinous tissue, the additional ingredients should be suitable for application to keratinous tissue, that is, when incorporated into the composition they are suitable for use in contact with human keratinous tissue (hair, nails, skin, lips) without undue toxicity, incompatibility, instability, allergic response, and the like within the scope of sound medical judgment. The CTFA Cosmetic Ingredient Handbook, Ninth Edition (2002) describes a wide variety of nonlimiting cosmetic and pharmaceutical ingredients commonly used in the skin care industry, which are suitable for use as additional ingredients in the compositions of the present invention. Non-limiting examples of these additional ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, thickeners, vitamins and derivatives thereof, hair straightening agents, hair coloring agents, hair bleaching agents, and combinations thereof.

In any embodiment of the present invention, however, the additional ingredients useful herein can be categorized by the benefit they provide or by their postulated mode of action. However, it is to be understood that the additional ingredients useful herein can in some instances provide more than one benefit or operate via more than one mode of action. Therefore, classifications herein are made for the sake of convenience and are not intended to limit the additional ingredients to that particular application or applications listed.

### Cosmetic or Personal Care Products

The compositions of the invention may be used in various cosmetic and/or personal care products, for example, skin care, hair care, nail care, facial and body care and sunscreen compositions, such as lotions, gels, sprays, and the like, hand cleaners, bath compositions, suntan oils, anti-perspirant compositions, perfumes and colognes, cold creams, hair sunscreen compositions, pre-shaves, deodorants, topical pharmaceutical ointments, skin moisturizers, facial cleansers, cleansing creams, skin gels, shampoos, hair conditioners, detergents, household cleaning products, make-up products, lipstick products, mascara, hair coloring products, hair straightening agents, hair coloring agents, hair bleaching agents, and combinations thereof. Therefore, in addition to any of the above cited skin care or hair care peptides and other actives, the cosmetic compositions described in the present invention may often include as an additional ingredient a dermatologically acceptable carrier. The form of the composition and the final product may be any of the following: liquids, gels, creams, water-in-oil and oil-in-water, and silicone emulsions, foams, and solids; they may be clear or opaque; and may be formulated as both aqueous and non-aqueous preparations, including but not limited to topical preparations.

### Packaging/Treatment Kits

The present invention relates to a kit for conveniently and effectively carrying out the methods in accordance with the present invention. Such kits may be suited for the delivery of solid oral forms such as tablets or capsules. Such a kit may include a number of unit dosages. Such kits can include a means for containing the dosages oriented in the order of their intended use. An example of a means for containing the dosages in the order of their intended uses is a card. An example of such a kit is a “blister pack”. Blister packs are well known in the packaging industry and are widely used for packaging pharmaceutical unit dosage forms. If desired, the blister can be in the form of a childproof blister, i.e., a blister that is difficult for a child to open, yet can be readily opened by an adult. If desired, a memory aid can be provided, for example in the form of numbers, letters, or other markings or with a calendar feature and/or calendar insert, designating the days and the sections of a day in the treatment schedule in which the dosages can be administered, such as an AM dose is packaged with a “mid day” and a PM dose; or an AM dose is packaged with a PM dose. Alternatively, placebo dosages, or vitamin or dietary supplements, either in a form similar to or distinct from the pharmaceutical active dosages, can be included.

In one aspect, the package, kit or container comprises a “blister package” (also called a blister pack, or bubble pack). In one aspect, the blister package consists two or more separate compartments: Am dosage of this invention, and PM dosage of this invention, or mid-day dosage of this invention. This blister package is made up of two separate material elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, the invention provides for blister packs, clamshells or trays comprising a composition (e.g., a (the multi-ingredient combination of drugs of the invention) combination of active ingredients) of the invention. Blister packs, clamshells or trays can be designed to be non-reclosable, so consumers can tell if a package has already opened. They are used to package for sale goods where product tampering is a consideration, such as the pharmaceuticals of the invention. In one aspect, a blister pack of the invention comprises a moulded PVC base, with raised areas (the “blisters”) to contain the tablets, pills, etc. comprising the combinations of the invention, covered by a foil laminate. Tablets, pills, etc. are removed from the pack either by peeling the foil back or by pushing the blister to force the tablet to break the foil. In one aspect, a specialized form of a blister pack is a strip pack.

In one aspect, a blister pack also comprises a method of packaging where the compositions comprising combinations of ingredients of the invention are contained in-between a card and a clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. of the invention are used, and they can comprise of two vacuum-formed PVC sheets meshed together at the edges, with the informative card inside.

In one aspect, blister packaging comprises at least two components (e.g., is a multi-ingredient combination of drugs of the invention): a thermoformed “blister” which houses the product (e.g., a pharmaceutical combination of the invention), and then a “blister card” that is a printed card with an adhesive coating on the front surface. During the assembly process, the blister component, which is most commonly made out of PVC, is attached to the blister card using a blister machine. This machine introduces heat to the flange area of the blister which activates the glue on the card in that specific area and ultimately secures the PVG blister to the printed blister card. The thermoformed PVG blister and the printed blister card can be as small or large. Conventional blister packs can also be sealed (e.g., using an AERGO 8 DUO®, SCA Consumer Packaging, Inc., DeKalb, Ill.) using regular heat seal tooling. This alternative aspect, using heat seal tooling, can seal common types of thermoformed packaging.

As discussed herein, the products of manufacture of the invention can comprise the packaging of the therapeutic drug combinations of the invention, alone or in combination, as “blister packages” or as a plurality of packettes, including as lidded blister packages, lidded blister or blister card or packets, or a shrink wrap.

In one aspect, laminated aluminum foil blister packs are used, e.g., for the preparation of drugs designed to dissolve immediately in the mouth of a patient. This exemplary process comprises having the drug combinations of the invention prepared as an aqueous solution(s) which are dispensed (e.g., by measured dose) into an aluminum (e.g., alufoil) laminated tray portion of a blister pack. This tray is then freeze-dried to form tablets which take the shape of the blister pockets. The alufoil laminate of both the tray and lid fully protects any highly hygroscopic and/or sensitive individual doses. In one aspect, the pack incorporates a child-proof peel open security laminate. In one aspect, the system give tablets an identification mark by embossing a design into the alufoil pocket that is taken up by the tablets when they change from aqueous to solid state. In one aspect, individual push-through blister packs/packettes are used, e.g., using hard temper aluminum (e.g., alufoil) lidding material. In one aspect, hermetically-sealed-high barrier aluminum (e.g., alufoil) laminates are used. In one aspect, any of the invention's products of manufacture, including kits or blister packs, use foil laminations and strip packs, stick packs, sachets and pouches, peelable and non-peelable laminations combining foil, paper, and film for high barrier packaging.

Other means for containing said unit dosages can include bottles and vials, wherein the bottle or vial comprises a memory aid, such as a printed label for administering said unit dosage or dosages. The label can also contain removable reminder stickers for placement on a calendar or dayminder to further help the patient to remember when to take a dosage or when a dosage has been taken.

### Formulations for Alternate Specific Routes of Administration

The pharmaceutical compositions may be optimized for particular types of delivery. For example, pharmaceutical compositions for oral delivery are formulated using pharmaceutically acceptable carriers that are well known in the art. The carriers enable the agents in the composition to be formulated, for example, as a tablet, pill, capsule, solution, suspension, sustained release formulation; powder, liquid or gel for oral ingestion by the subject.

The GR antagonist may also be delivered in an aerosol spray preparation from a pressurized pack, a nebulizer or from a dry powder inhaler. Suitable propellants that can be used in a nebulizer include, for example, dichlorodifluoro-methane, trichlorofluoromethane, dichlorotetrafluoroethane and carbon dioxide. The dosage can be determined by providing a valve to deliver a regulated amount of the compound in the case of a pressurized aerosol.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. Preferably the compositions are administered by the oral, intranasal or respiratory route for local or systemic effect. Compositions in preferably sterile pharmaceutically acceptable solvents may be nebulized by use of inert gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices that deliver the formulation in an appropriate manner.

### Administration

Typically, the composition may be applied repeatedly for a sustained period of time topically on the part of the body to be treated, for example, the eyelids, eyebrows, skin or scalp. The dosage regimen will generally involve regular, such as daily, administration for a period of treatment of at least one month, or at least three months, or at least six months.

Alternatively, the composition may be applied intermittently, or in a pulsed manner. Accordingly, an alternative embodiment of the invention is to apply the composition on an intermittent or pulsed dosage schedule. For example, the composition of the invention may be used for two or more days, stopped, then restarted again at a time from between 2 weeks to 3 months later, and at even more long-spaced intervals in the case of the scalp.

For topical use on the eyelids or eyebrows, the composition can be formulated in aqueous solutions, creams, ointments or oils exhibiting physiologically acceptable osmolarity by addition of pharmacologically acceptable buffers and salts. Such formulations may or may not, depending on the dispenser, contain preservatives such as benzalkonium chloride, chlorhexidine, chlorobutanol, parahydroxybenzoic acids and phenylmercuric salts such as nitrate, chloride, acetate, and borate, or antioxidants, as well as additives like EDTA, sorbitol, boric acid etc. as additives. Furthermore, particularly aqueous solutions may contain viscosity increasing agents such as polysaccharides, e.g., methylcellulose, mucopolysaccharides, e.g., hyaluronic acid and chondroitin sulfate, or polyalcohol, e.g., polyvinylalcohol. Various slow releasing gels and matrices may also be employed as well as soluble and insoluble ocular inserts, for instance, based on substances forming in-situ gels. Depending on the actual formulation and hair growth stimulant, and/or hair growth stimulating agent and/or hair density increasing agent and/or hair loss prevention agent to be used, various amounts of the drug and different dose regimens may be employed. Typically, the daily amount of the agent of the invention and/or hair density increasing agent and/or hair loss prevention agent for treatment may be about 0.1 ng to about 100 mg, or about 1 ng to about 100 μg.

For topical use on the skin and the scalp, the composition can be advantageously formulated using ointments, creams, liniments or patches as a carrier of the active ingredient. Also, these formulations may or may not contain preservatives, depending on the dispenser and nature of use. Such preservatives include those mentioned above, and methyl-, propyl-, or butyl-parahydroxybenzoic acid, betain, chlorhexidine, benzalkonium chloride, and the like. Various matrices for slow release delivery may also be used. Typically, the dose to be applied is in the range of about 0.1 ng to about 100 mg per day, or about 1 ng to about 10 mg per day, or about 10 ng to about 1 mg per day depending on the hair growth stimulant, and/or hair growth stimulating agent and/or hair density increasing agent and/or hair loss prevention agent and the formulation. To achieve the daily amount of medication depending on the formulation, the hair growth stimulant, and/or hair growth stimulating agent and/or hair density increasing agent and/or hair loss prevention agent may be administered once or several times daily with or without antioxidants.

What is claimed is:

1. A topical composition for treating or preventing stress induced hair loss in a patient comprising:
a hair growth stimulating agent which is at least one GR antagonist in a therapeutically effective amount;
a cosmetically or pharmaceutically acceptable excipient, carrier or vehicle,
wherein the composition has the effect of treating or preventing stress induced hair loss in the patient.

2. The composition of claim 1, wherein the GR antagonist is selected from the group consisting of ORG 34517, 11-(substituted phenyl)-estra-4,9-diene derivatives, and 11-(substituted phenyl)-estra-4,9-diene derivatives of formula I 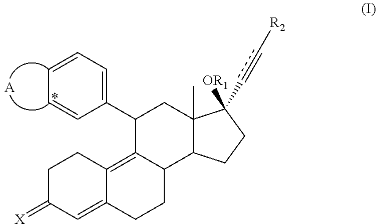
wherein A is a residue of a 5- or 6-membered ring containing 2 heteroatoms which are not connected to each other and independently selected from O and S, the ring being optionally substituted with one or more halogen atoms, or A is a residue of a 5- or 6-membered ring wherein no double C—C bonds are present, containing 1 heteroatom selected from O and S, which heteroatom is connected to the phenyl group at the position indicated with an asterisk, the ring being optionally substituted with one or more halogen atoms; R1 is H or 1-oxo(1-4C)alkyl; R2 is H, (1-8C)alkyl, halogen or CF3; X is selected from (H,OH), O, and NOH; and the interrupted line represents an optional bond, and combinations thereof.

3. The composition of claim 1, wherein the hair is selected from the group consisting of head hair, scalp hair, eyelashes, eyebrows, mustaches, beards, chest, legs, arms, and combinations thereof.

4. The composition of claim 1 in a form selected from the group consisting of application stick, mascara, eyebrow coloring, eyeliner, eye shadow or other eye lid cosmetics, facial or body powder, sun tanning lotions and creams and sprays, sunscreens and blocks, nail polish, mousse, sprays, styling gels, nail conditioner, whether in the form of creams, lotions, gels, ointments, emulsions, colloids, solutions, suspensions, compacts, solids, pencils, brushes, spray-on formulations, brush-on formulations and the like, bath and shower gels, shampoos, conditioners, cream rinses, hair dyes and coloring products, leave-on conditioners, lip balms, skin conditioners, hair sprays, soaps, body scrubs, exfoliants, astringents, depilatories and permanent or temporary waving solutions, antidandruff formulations, antisweat and antiperspirant compositions, shaving, preshaving and after shaving products, moisturizers, deodorants, cold creams, cleansers, skin gels, rinses, whether in solid, powder, liquid, cream, gel, ointment, lotion, emulsions, colloids, solutions, suspensions, or other form, gels, patches, creams, sprays, ointments, lotions, emulsions, colloids, solutions, suspensions, powders and the like, and combinations thereof.

5. The composition of claim 1, further comprising at least one other ingredient selected from the group consisting of solvents, thickeners or gelling agents, coloring materials which are soluble in the medium of the composition, fillers, pigments, antioxidants, preservatives, fragrances, electrolytes, neutralizing agents, film-forming polymers, UV blocking agents, cosmetic and pharmaceutical active principles other than GR antagonists, and combinations thereof.

6. The composition of claim 5, wherein said at least one additional ingredient is a cleaning agent, hair conditioning agent, skin conditioning agent, hair styling agent, antidandruff agent, hair growth promoter, perfume, sunscreen, sunblock, pigment, moisturizer, film former, hair color, make-up agent, detergent, pharmaceutical, thickening agent, emulsifier, humectant, emollient, antiseptic agent, deodorant active, dermatologically acceptable carrier, surfactant, abrasive, absorbent, fragrance, coloring/colorant, essential oil, skin sensate, astringent, anti-acne agent, anti-caking agent, antifoaming agent, antimicrobial, antioxidant, binder, biological additive, enzyme, enzyme inhibitor, enzyme activator, coenzyme, botanical extract, ceramide, addition peptide, buffering agent, bulking agent, chelating agent, cosmetic biocide, denaturant drug astringent, external analgesic, polymer, quat, substantivity increasing agent, opacifying agent, pH adjuster, propellant, reducing agent, sequestrant, skin bleaching agent, skin lightening agent, skin-conditioning agent, skin soothing agent, skin healing agent, aloe vera, pantothenic acid and derivative thereof, allantoin, bisabolol, dipotassium glycyrrhizinate, skin treating agent, thickener, or a vitamin or derivative thereof and mixtures or combinations thereof.

7. The composition of claim 1 further comprising a plurality of additional ingredients and wherein at least one of said additional ingredients is a dermatologically acceptable carrier.

8. A delivery device for the targeted application to the hair of a mammal a therapeutically effective amount of the composition of claim 1.

9. The delivery device of claim 8, wherein the device is for the application of the composition to eyelashes, eyebrows, and/or scalp.

10. A method for treating or preventing stress induced hair loss wherein said method comprises administering, to a patient in need of such therapy, at least one GR antagonist in a therapeutically effective amount.

11. The method, according to claim 10, wherein the at least one GR antagonist is in a pharmaceutical preparation.

12. The method of claim 10, wherein the GR antagonist is selected from the group consisting of ORG 34517, 11-(substituted phenyl)-estra-4,9-diene derivatives, and 11-(substituted phenyl)-estra-4,9-diene derivatives of formula I 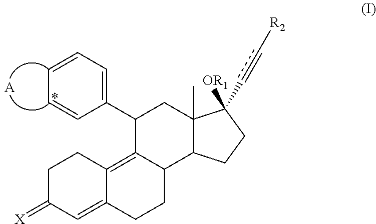
wherein A is a residue of a 5- or 6-membered ring containing 2 heteroatoms which are not connected to each other and independently selected from O and S, the ring being optionally substituted with one or more halogen atoms, or A is a residue of a 5- or 6-membered ring wherein no double C—C bonds are present, containing 1 heteroatom selected from 0 and 5, which heteroatom is connected to the phenyl group at the position indicated with an asterisk, the ring being optionally substituted with one or more halogen atoms; R1 is H or 1-oxo(1-4C)alkyl; R2 is H, (1-8C)alkyl, halogen or CF3; X is selected from (H,OH), O, and NOH; and the interrupted line represents an optional bond.

13. The method of claim 10, wherein the hair is selected from the group consisting of head hair, scalp hair, eyelashes, eyebrows, mustaches, beards, chest, legs, arms, and combinations thereof.

14. A method of treating or preventing hair loss secondary to stress in a patient in need thereof, comprising administering at least one GR antagonist in a therapeutically effective amount.

15. The method of claim 14, wherein the stress is selected from the group consisting of chemotherapy, traumatic events, and combinations thereof.

16. The method of claim 14, wherein the hair is selected from the group consisting of head hair, scalp hair, eyelashes, eyebrows, mustaches, beards, chest, legs, arms, and combinations thereof.

17. A kit for treating or preventing stress induced hair loss comprising:
(a) a pharmaceutical composition comprising at least one GR antagonist in a therapeutically effective amount; and
(b) at least one blister package; a lidded blister; a blister card or packet; a clamshell; an intravenous (IV) package, IV packette or IV container; a tray or a shrink wrap comprising the pharmaceutical composition of (a) and instructions for use of the pharmaceutical composition for treating or preventing stress induced hair loss.

18. A product of manufacture for treating or preventing stress induced hair loss comprising a blister package; a lidded blister; a blister card or packet; a clamshell; an intravenous (IV) package, IV packette or IV container; a tray or a shrink wrap comprising a pharmaceutical composition comprising at least one GR antagonist, and instructions for use of the pharmaceutical composition for treating or preventing stress induced hair loss.

19. A topical composition for treating or preventing stress induced keratinous tissue damage in a patient comprising:
a keratinous tissue growth stimulating agent which is at least one GR antagonist in a therapeutically effective amount;
a cosmetically or pharmaceutically acceptable excipient, carrier or vehicle,
wherein the composition has the effect of treating or preventing stress induced keratinous tissue damage in the patient.

20. The composition of claim 19, wherein the GR antagonist is selected from the group consisting of ORG 34517, 11-(substituted phenyl)-estra-4,9-diene derivatives, and 11-(substituted phenyl)-estra-4,9-diene derivatives of formula I 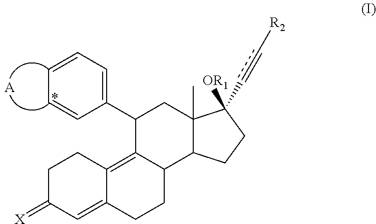
wherein A is a residue of a 5- or 6-membered ring containing 2 heteroatoms which are not connected to each other and independently selected from O and S, the ring being optionally substituted with one or more halogen atoms, or A is a residue of a 5- or 6-membered ring wherein no double C—C bonds are present, containing 1 heteroatom selected from O and S, which heteroatom is connected to the phenyl group at the position indicated with an asterisk, the ring being optionally substituted with one or more halogen atoms; R1 is H or 1-oxo(1-4C)alkyl; R2 is H, (1-8C)alkyl, halogen or CF3; X is selected from (H,OH), O, and NOH; and the interrupted line represents an optional bond, and combinations thereof.

21. The composition of claim 19, wherein the keratinous tissue is selected from the group consisting of toenails, fingernails, cuticles, claws, hooves, and combinations thereof.

22. The composition of claim 19 in a form selected from the group consisting of application stick, mascara, eyebrow coloring, eyeliner, eye shadow or other eye lid cosmetics, facial or body powder, sun tanning lotions and creams and sprays, sunscreens and blocks, nail polish, mousse, sprays, styling gels, nail conditioner, whether in the form of creams, lotions, gels, ointments, emulsions, colloids, solutions, suspensions, compacts, solids, pencils, brushes, spray-on formulations, brush-on formulations and the like, bath and shower gels, shampoos, conditioners, cream rinses, hair dyes and coloring products, leave-on conditioners, lip balms, skin conditioners, hair sprays, soaps, body scrubs, exfoliants, astringents, depilatories and permanent or temporary waving solutions, antidandruff formulations, antisweat and antiperspirant compositions, shaving, preshaving and after shaving products, moisturizers, deodorants, cold creams, cleansers, skin gels, rinses, whether in solid, powder, liquid, cream, gel, ointment, lotion, emulsions, colloids, solutions, suspensions, or other form, gels, patches, creams, sprays, ointments, lotions, emulsions, colloids, solutions, suspensions, powders and the like, and combinations thereof.

23. The composition of claim 19, further comprising at least one other ingredient selected from the group consisting of solvents, thickeners or gelling agents, coloring materials which are soluble in the medium of the composition, fillers, pigments, antioxidants, preservatives, fragrances, electrolytes, neutralizing agents, film-forming polymers, UV blocking agents, cosmetic and pharmaceutical active principles other than GR antagonists, and combinations thereof.

24. The composition of claim 23, wherein said at least one additional ingredient is a cleaning agent, hair conditioning agent, skin conditioning agent, hair styling agent, antidandruff agent, hair growth promoter, perfume, sunscreen, sunblock, pigment, moisturizer, film former, hair color, make-up agent, detergent, pharmaceutical, thickening agent, emulsifier, humectant, emollient, antiseptic agent, deodorant active, dermatologically acceptable carrier, surfactant, abrasive, absorbent, fragrance, coloring/colorant, essential oil, skin sensate, astringent, anti-acne agent, anti-caking agent, antifoaming agent, antimicrobial, antioxidant, binder, biological additive, enzyme, enzyme inhibitor, enzyme activator, coenzyme, botanical extract, ceramide, addition peptide, buffering agent, bulking agent, chelating agent, cosmetic biocide, denaturant drug astringent, external analgesic, polymer, quat, substantivity increasing agent, opacifying agent, pH adjuster, propellant, reducing agent, sequestrant, skin bleaching agent, skin lightening agent, skin-conditioning agent, skin soothing agent, skin healing agent, aloe vera, pantothenic acid and derivative thereof, allantoin, bisabolol, dipotassium glycyrrhizinate, skin treating agent, thickener, or a vitamin or derivative thereof and mixtures or combinations thereof.

25. The composition of claim 24 further comprising a plurality of additional ingredients and wherein at least one of said additional ingredients is a dermatologically acceptable carrier.

26. A delivery device for the targeted application to the hair of a mammal a therapeutically effective amount of the composition of claim 19.

27. The delivery device of claim 26, wherein the device is for the application of the composition to toenails, fingernails, cuticles, claws, hooves, and combinations thereof.

28. A method for treating or preventing stress induced keratinous tissue damage wherein said method comprises administering, to a patient in need of such therapy, at least one GR antagonist in a therapeutically effective amount.

29. The method, according to claim 28, wherein the at least one GR antagonist is in a pharmaceutical preparation.

30. The method of claim 28, wherein the GR antagonist is selected from the group consisting of ORG 34517, 11-(substituted phenyl)-estra-4,9-diene derivatives, and 11-(substituted phenyl)-estra-4,9-diene derivatives of formula I 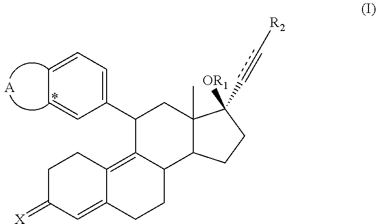
wherein A is a residue of a 5- or 6-membered ring containing 2 heteroatoms which are not connected to each other and independently selected from O and S, the ring being optionally substituted with one or more halogen atoms, or A is a residue of a 5- or 6-membered ring wherein no double C—C bonds are present, containing 1 heteroatom selected from O and S, which heteroatom is connected to the phenyl group at the position indicated with an asterisk, the ring being optionally substituted with one or more halogen atoms; R1 is H or 1-oxo(1-4C)alkyl; R2 is H, (1-8C)alkyl, halogen or CF3; X is selected from (H,OH), O, and NOH; and the interrupted line represents an optional bond.

31. The method of claim 28, wherein the hair is selected from the group consisting of toenails, fingernails, cuticles, claws, hooves, and combinations thereof.

32. A method of treating or preventing keratinous tissue damage secondary to stress in a patient in need thereof, comprising administering at least one GR antagonist in a therapeutically effective amount.

33. The method of claim 32, wherein the stress is selected from the group consisting of chemotherapy, traumatic events, and combinations thereof.

34. The method of claim 32, wherein the hair is selected from the group consisting of toenails, fingernails, cuticles, claws, hooves, and combinations thereof.

35. A kit for treating or preventing stress induced keratinous tissue damage comprising:
(a) a pharmaceutical composition comprising at least one GR antagonist in a therapeutically effective amount; and
(b) at least one blister package; a lidded blister; a blister card or packet; a clamshell; an intravenous (IV) package, W packette or IV container; a tray or a shrink wrap comprising the pharmaceutical composition of (a) and instructions for use of the pharmaceutical composition for treating or preventing stress induced keratinous tissue damage.

36. A product of manufacture for treating or preventing stress induced keratinous tissue damage comprising a blister package; a lidded blister; a blister card or packet; a clamshell; an intravenous (IV) package, IV packette or IV container; a tray or a shrink wrap comprising a pharmaceutical composition comprising at least one GR antagonist, and instructions for use of the pharmaceutical composition for treating or preventing stress induced keratinous tissue damage.
